(19) United States
(12) Patent Application Publication
(10) Pub. No.: US 2023/0310710 A1
(43) Pub. Date: Oct. 5, 2023

(54) METHOD OF GENERATING A DRIED DRUG FORMULATION

(71) Applicant: HOFFMANN-LA ROCHE, INC., Little Falls, NJ (US)

(72) Inventors: Daniel KULLMANN, Basel (CH); Carmen LEMA MARTINEZ, Basel (CH); Sima RAHIMIAN, Basel (CH); Joerg LUEMKEMANN, Basel (CH); Gregoire SCHWACH, Basel (CH); Zana MARIN, Basel (CH)

(21) Appl. No.: 17/996,708

(22) PCT Filed: Apr. 19, 2021
(86) PCT No.: PCT/EP2021/060120

§ 371 Date: Oct. 20, 2022

(30) Foreign Application Priority Data
Apr. 20, 2020 (EP) 20170278.4

(51) Int. Cl. A61L 27/36; A61L 27/54; A61L 27/56

(52) U.S. Cl. CPC A61L 27/3691; A61L 27/54; A61L 27/56; B33Y 10/00

(57) ABSTRACT

A method of generating a lyophilized drug formulation (2) comprises: preparing a substrate to be additive manufacturable, wherein the substrate comprises a drug; additive manufacturing the substrate such that a scaffold body (21) is formed of the substrate; and lyophilizing the scaffold body (21).

### TECHNICAL FIELD

The present invention relates to a method of generating a dried or lyophilized drug formulation. Such methods allow for efficiently providing the drug formulation in a comparably stable state.

### BACKGROUND ART

In many pharmaceutical and other applications substances are provided in a dry form. Thereby, in order to achieve a long shelf life, it is often aimed to have the substances as dry as possible. In this context, it is known to desiccate or freeze dry the substances. Particularly, where it is necessary or beneficial to gently drying the substances, e.g. without heating them in an inappropriate manner, lyophilisation often is preferred.

For example, in pharmaceutical applications, frequently drug substances are to be, e.g., orally, parenterally, intravenously or subcutaneously, administered in liquid form. As an example, for intravenous administration it is known to use infusion bags which can be hanged on a support and continuously drip a liquid drug substance or a drug-diluent mixture through an infusion needle into a patient. However, in connection with liquid drug substances, many pharmaceuticals and particularly biopharmaceuticals cannot be stored and supplied for an appropriate duration in liquid form since they are commonly not sufficiently stable in that form. More specifically, many antibiotics and biological drugs are unstable in liquid form such that their quality cannot be maintained in a liquid state. In particular, stress caused by shaking, microbiological growth, aggregation or the like may compromise the drugs. As mentioned, it is however known to supply the drug in a dry form, such as in a powder or the like, in which they are essentially more stable and robust compared to the liquid form. The dry drug substance or formulation is then reconstituted or dissolved shortly before administration.

To achieve or maintain appropriate hygienic and quality standards the substances are often carefully lyophilised in specific conditions such as an aseptic environment. Thereby, the substances are usually lyophilised in specific containers in which they are positioned on shelfs of freeze dryers in aseptic conditions. After lyophilisation, the substances in the containers are either stored within these containers or transferred to other containers or packages for storage and supply. Shortly before being applied or administered the substances are then reconstituted.

Particularly, lyophilisation in aseptic environments often is effortful and time consuming resulting in comparably costly or low economic processes. Therefore, there is a need for a system or process allowing to increase efficiency of manufacturing or generating dried or lyophilized drug products or creating dry drug formulations in a continuous manner.

### DISCLOSURE OF THE INVENTION

According to the invention this need is settled by a method of generating a dried drug formulation as it is defined by the features of independent claim 1. Preferred embodiments are subject of the dependent claims.

In particular, in one aspect the invention is a method of generating or manufacturing a dried drug formulation. The method comprises a step of preparing a substrate to be additively manufactured, wherein the substrate comprises a drug, a step of additive manufacturing the substrate such that a scaffold body is formed of the substrate, and a step of drying the scaffold body. The result of this drying is the dried drug formulation provided as a scaffold body.

The drying in the context of the scaffold body and, thus, the drug formulation relates to specific procedures of removing humidity, i.e. water or another solvent. More specifically, since typically drug containing substances require a gentle processing for preventing any impairment of the drug, advantageously drying in the context of the invention involves either lyophilization or desiccation.

Lyophilisation in this context is a low temperature dehydration process, which involves freezing a substrate, lowering pressure and then removing ice by sublimation and desorption. The result from lyophilisation is the lyophilisate. Lyophilisation is also referred to as freeze drying. Lyophilisation can cover bulk freeze drying or even continuous freeze drying. Energy or heat transfer in lyophilisation is typically driven by convection and radiation processes wherein, in the specific process according to the invention, conduction may also be considerably important.

Desiccation in the context of the present invention is an ambient or slightly elevated temperature dehydration process, which involves the product to dehydrate in a continuous manner either under a constant temperature or within temperature cycles. The product is therefore transported on a conveyor band or tunnel and receives energy input for the progression of the desiccation process by convection via warm air or radiation via infrared sources.

The substrate can be any substance intended to undergo drying such as lyophilisation or desiccation. It can particularly be a liquid or fluid substance. Thereby, the term “fluid” relates to the property of the substrate to flow. Such fluid substrates can be liquid, granular, pasty or the like. The dried substance or dried scaffold body can, e.g., be intended to be consumed by a person by drinking, eating or the like. Preferably, the dried scaffold body is a lyophilised or desiccated drug formulation.

The term “drug” as used herein relates to a therapeutically active agent, also commonly called active pharmaceutical ingredient (API), as well as to a combination of plural such therapeutically active substances. The term also encompasses diagnostic or imaging agents, like for example contrast agents (e.g. MRI contrast agents), tracers (e.g. PET tracers) and hormones, that need to be administered in liquid form to the patient.

The term “drug formulation” as used herein relates to a single drug as defined above or a plurality of such drugs mixed or formulated. For example, besides the drug, a drug formulation may additionally comprise an excipient and/or other auxiliary ingredients. When being a dry drug formulation, the dried scaffold body can be a solid drug formulation, a semisolid or powderous drug substance.

The term “drug substance” as used herein relates to a drug formulation as defined above in a form that is suitable for administration to the patient. Thereby, the drug substance can be the pure drug formulation or a drug formulation reconstituted, diluted or dissolved in an administrable form. A particularly preferred drug substance in the context of the invention is a solution, in particular a solution for oral, parenteral intrathecal or ophthalmic administration, injection or infusion.

The term “drug product” as used herein relates to a finished end product comprising a drug substance or a plurality of drug substances. In particular, a drug product may be a ready to use product having the drug substance in an appropriate dosage and/or in an appropriate form for administration. For example, a drug product may include a handling or storage device such as a flexible container.

The term “potency” used in connection with the drug formulation can be a measure of drug activity expressed in terms of the amount required to produce an effect of given intensity. Thus, the terms “high potency”, “highly potent” or similar can relate to a formulation or substance which is active at comparably small amounts or dosages. In other words, a highly potent drug formulation can evoke a given response at comparably low concentration, while a drug formulation of lower potency can evoke the same response only at higher concentrations. The potency may depend on both the affinity and efficacy of the drug formulation. Handling such drug formulations or substances can be particularly challenging since comparably small variations in dosing can be detrimental.

In numbers, a highly potent drug formulation can be defined as a drug formulation having a biological activity at approximately 15 micrograms (μg) per kilogram (kg) of body weight or below in humans. This is equivalent to a therapeutic dose at approximately 1 milligrams (mg) or below in humans. The highly potent drug formulation can thus be defined as a drug having an inhalative Acceptable Daily Exposure (ADE) value of 1.5 μg/d or less, translating into an Indicative Occupational Exposure Limit (IOEL) value of 0.15 μg/m³. In particular, the highly potent drug formulation can be a class 3B drug or the like. When used with highly potent drug formulations to be administered by infusion, the method according to the invention can be particularly beneficial.

Preferably, the drug comprises a protein, wherein the protein can be an antibody such as a monoclonal antibody. When using such a protein containing drug, the effects achieved by the method according to the invention can be particularly advantageous. Furthermore, compared to conventional protein drug formulation involving lyophilization from a liquid form, in accordance with the invention, stability can particularly be increased when protein drugs are involved.

The scaffold body typically is a three dimensional scaffold body. The term “three dimensional” in this connection can relate to extensions in all three directions in space. Thereby, compared to two dimensional bodies, such as sheet-like or plate-like bodies, which do not essentially extend in one direction in space, three dimensional bodies do have a spatial dimension.

The different steps of the method can be performed in or by one single device or, more preferably, in or by plural devices allowing for a specifically suitable implementation, even though the substrate or drug formulation or scaffold body has to be transferred from one device to the other device. For example, the additive manufacturing can be executed by a specific apparatus or equipment. Further, the drying of the scaffold body can be performed in or by a freeze dryer. Like this, an efficient execution of each step of the method can be achieved.

Preparing the substrate can particularly result in provision of the substrate in a form that it can be used by the apparatus or equipment executing the additive manufacturing. For example, the substrate can be prepared to have specific properties such as composition, viscosity, granularity or the like suitable for the involved additive manufacturing.

By additive manufacturing the drug formulation, it can be provided in a shape particularly suitable for the specific drying applied and for providing a comparably robust dried drug formulation. Thereby, the shape can be designed to allow a particularly efficient heat transfer to the complete drug formulation or substrate or a specific design to allow particularly effective liquid vapor removal. For example, the drug formulation provided for the lyophilization can be embodied in a shape to have a comparably large heat transfer surface such provided with tunnels for vapor removal.

By the method according to the invention, a comparably low duration for drying the drug formulation can be reached. In particular, it can be achieved that heat is efficiently transferred to the drug formulation and that water vapor or other vapor is efficiently transferred away from the drug formulation such that a comparably fast drying of the drug formulation is possible. For achieving this, the substrate can be designed and shaped such that it has a comparably large contact and drying surface allowing for transferring heat to and vapor from the substrate. The efficiency of the dehydration process can be increased and, thus, the occupancy rate of the freeze dryer too. Also, due to the specific shape and the increased sublimation area provided by the additive manufacturing, and the reduction in product-shelf-distance, it can be achieved that the substrate is homogeneously dried. Furthermore, by designing a continuous additive manufacturing process the dehydration in a desiccation tunnel with a continuous conveyer belt can also lead to a more homogeneous and efficient drying of the scaffold body.

Furthermore, by allowing the dried drug formulation to be in a dedicated shape, the drug formulation can efficiently be reconstituted before administration. For example, the shape of the dried drug formulation can be designed to allow a diluent to efficiently cover and penetrate the entire structure and dissolve the drug formulation before administration in order to generate a drug substance. Like this, it can be assured that the drug formulation is completely dissolved allowing for a proper administration. The need for actively dissolving the drug formulation, e.g. by shaking or stirring, which may harm the drug, can be reduced.

Still further, the shape of the dried substrate or drug formulation can be designed to allow visual recognition of the drug contained and/or of its dosage in the dried substrate or scaffold body. This allows for an efficient handling of the final drug formulation. Also, the shape can allow a robust provision of the dried drug formulation. Further, it allows for providing a counterfeit identifier. By 3D printing several safety measures can be included into the created structure, i.e. the scaffold body, to ensure the identification of the proper product. For example, an engraving like pattern can be embodied in the scaffold body.

The term “additive manufacturing” (AM) in the context of the invention generally relates to building three dimensional objects by adding material stepwise until the objects are finalized. Typically, the material is added layer-upon-layer and, thereby, the object is generated. However, the object can also be generated by a choice of different materials. For example, the different materials can comprise different drugs to be combined only upon administration.

The additive manufacturing can be a process of extruding the substrate, of binder dispensing on the substrate, of binder spraying on the substrate, of paste extrusion modelling the substrate, of fuse deposition modelling the substrate, of laminated object manufacturing the substrate, of using stereo lithography to generate the substrate, or the like.

Advantageously, the dried scaffold body is used or provided as drug substance without any further adaption or change of the dried scaffold body. In particular, the method can comprise a step of providing the dried scaffold body as drug substance, wherein such provision may involve a step of packaging the dried scaffold body. Like this, particularly when biological drugs or biopharmaceuticals are involved, which may comparably quickly deteriorate in a liquid state, stability and applicability of the drug substance in use can be increased.

The method according to the invention may further involve a step of designing the shape of the scaffold body to be created. Thereby, different properties or features can be embodied which may be beneficial in generation and application of the drug substance being the dried scaffold body. For example, the shape may be designed such that the scaffold body includes comparably wide pores which may act as chimneys in lyophilization or other drying procedure. Like this, drying may be particularly efficient and, specifically, removal of sublimed water may be improved. Also, a thinner cake distance between a sublimation front and a chamber environment may allow to provide less resistance matter for the water vapor to escape the cake or scaffold body. Direct contact of the cake material or scaffold body to the energy supplying shelf may improve the transfer of heat necessary for the continuation of the sublimation or drying process and, thus, may shorten process time. Conical shapes of the scaffold body being broad at the bottom and narrow at the top, or intricate inner geometries may allow improved heat flow while maintaining good removal of the water vapor and can facilitate an improved drying as well. For example, a dense or viscous material typically will require lower temperatures during lyophilization as higher cake resistance and therefore lower sublimation rates typically are involved. Also, cooler printing temperatures may be needed for solutions with lower viscosities to avoid running of the printed shape or geometry.

Preferably, the additive manufacturing comprises 3D printing the substrate. When 3D printing the substrate, it can be necessary to adapt parameters of the printing process to the properties of the substrate. For example, layer thickness, numbers of layers or pore diameter, the type of print head, printing speed and/or temperature can be adapted in accordance with the substrate involved. 3D printing allows to create a huge variety of shapes of the substrate. Thereby, the freedom of designing the shape to achieve efficient drying can be comparably high.

Preparing the substrate preferably comprises generating a printing ink, printing paste or printing granulate of the substrate suitable for 3D printing. Such preparation may involve defining particle size, viscosity, charge ratio, melting temperature, mixing rate and/or drug concentration

Preferably, the scaffold body has pores. Thereby, the pores can be through holes or passageways in the interior of the scaffold body. They can form a network of ducts inside the scaffold body which is accessible through openings at a circumference or boundary of the scaffold body. Such pores allow vapor to be transferred during the drying process away from the substrate. Also, pores allow for an efficient reconstitution of the drug formulation embodied by the substrate in the scaffold body.

Thereby, the scaffold body preferably has end sides and each of the pores of the scaffold has an opening at one of the end sides. The pores of the scaffold body preferably are passageways extending between at least two of the end sides of the scaffold body. Such passageways opening at an end side or, in particular, connecting two end sides allow for an efficient heat and/or vapor transfer through the scaffold body.

The pores of the scaffold body can have a diameter in a range of about 1 mm to about 5 mm, of about 1 mm to about 1.2 mm, of about 1.8 mm to about 2.2 mm, or of about 3.5 mm to about 4.5 mm. Pores being dimensioned with such diameters are suitable for efficiently transferring heat into and vapor out of the substrate during drying.

Preferably, the scaffold body has a cylindrical shape. The cylindrical shape can be established by a cylinder having a circular, oval or polygonal base. Thereby, the base of the scaffold body or cylinder preferably has a diameter of in between about 15 mm to about 60 mm. Such cylinders allow for an efficient handling of the scaffold body. In particular, the cylinders can be dimensioned and shaped similarly as vials such that handling equipment of vials can be used for handling the substrate and the dried drug formulation. More specifically, a common freeze dryer embodied for vials can be used for lyophilization.

Preferably, the method comprises a step of gathering drying properties of the specific drying procedure applied when drying the scaffold body, designing a shape of the scaffold body in accordance with the gathered drying properties, and configuring the additive manufacturing of the substrate such that the scaffold body is formed of the substrate in the designed shape. Designing the shape of the scaffold body can be done on a computer such as, e.g., by means of a computer aided design (CAD) tool or a similar tool. Advantageously, a tool is used which allows to generate a dataset or g-code, i.e. layers of the dataset cut by a slicer program or the like, that can be transferred or imported by the instrument used for additive manufacturing the substrate such as a 3D printer or the like. Thus, the method can comprise a step of generating a dataset representing the shape of the scaffold body in layers, wherein the additive manufacturing of the substrate is configured by means of the dataset such that the scaffold body is formed of the substrate in the designed shape.

Preferably, the method comprises a step of gathering substrate properties of the substrate. Involving properties of the substrate when setting up and executing the method according to the invention allows for achieving a particularly suitable and sophisticated procedure.

Substrate properties important for additive manufacturing and, more specifically, to 3D-printing typically include viscosity and temperature of the substrate or ink and of the environment. When protein containing drugs are involved, viscosity usually is impacted by protein concentration.

For example, the additive manufacturing steps or printing process can be performed at room temperature as well as higher temperatures. However, to keep required or beneficial high viscosity values of the substrate or ink, lower temperatures are typically preferred. Specifically, in 3D-printing of protein containing substrates printing can be done at about 2° C. to about 8° C. and at a room temperature with the substrate kept on ice before printing. The printed scaffolds can then be returned to ice or frozen status after being printed. In cases of substrates involving viscosity decrease with increasing shear strain, viscosity can be in a range of between 100 mPas (milli-Pascal seconds) and 800 mPas, however, printing of much higher initial viscosities is also feasible. The range of viscosity relates to viscosities at a constant shear rate of 1'000 per second and a temperature at 20° C.

Thereby, the method preferably further comprises steps of designing a shape of the scaffold body in accordance with the gathered substrate properties, and configuring the additive manufacturing of the substrate such as the scaffold body is formed of the substrate in the designed shape. By involving the substrate properties, it can be achieved that an efficient drying is possible. For example, depending on the properties of the substrate, the surface accessible by heat in during lyophilization or desiccation, or the pore design in general can be optimized. Furthermore, a general passageway design of the scaffold body can be adapted to facilitate improved vapor exhaust due to the substrates' resistance properties.

Additionally or alternatively, the method preferably further comprises steps of selecting a printhead in accordance with the gathered substrate properties, and configuring the additive manufacturing of the substrate such the scaffold body is formed via the selected printhead. The printhead preferably is selected from the group of pneumatic printhead, piston printhead, screw printhead and electromagnetic droplet printhead.

When additive manufacturing and particularly 3D-printing the substrate containing the drug, selection of the type of the printhead allows to define how exactly the substrate is extruded. This directly impacts the printing parameters. These parameters are typically not all shared among different printing techniques/printheads as some are only applicable to the individual printing approach. Thus, selecting an appropriate printhead suiting the properties of the specific substrate allows for increasing efficiency of the process.

Common to all of types of printheads may be printing speed or the velocity with which the printhead moves. Nozzle/needle diameter through which the ink or substrate is extruded and layer thickness of any printed layer may be important parameters. If pneumatic printing is used, then the driving force is air pressure. In piston or screw driven extrusion, vertical or rotational mechanical forces may be applied, respectively. For pneumatic printhead the applied pressure is thus of importance and for piston/screw driven extrusion, the extrusion rate needs to be determined or predefined.

Additionally, inkjet or electromagnetic droplet printing can be used. In this case, small droplets are formed and deposited instead of an ink filament. This is achieved by opening and closing an electromagnetically actuated valve as the fluid is pressurized and it can flow when the valve is open. For this, the frequency of valve opening as well as the length of time for which the valve stays open need to be predefined or set. Legacy needles or nozzles cannot be used in this case.

Feasibility of printing with all the mentioned printheads may be efficient depending on the given circumstances such as, particularly the substrate parameters. E.g., for syringe pump type as piston printhead, wherein the substrate as ink is loaded in a syringe and extruded as the piston is forwarded, printing speeds from 1 mm/s to 20 mm/s combined with extrusion rates from 3 μL/s to 10 μL/s have proven to be appropriate, wherein needles or nozzles having a diameter in the range between 18 G to 22 G and a layer height or thickness in a range from 0.58 mm to 0.84 mm can be suitable. In the case where a high number of scaffolds is printed, e.g., for the purpose of stability study, the printing speed selected can be set to 3 mm/s and the extrusion rate to 1.9 can be suitable, wherein a 20 G diameter needle can be used. The 18 G, 20 G and 22 G may refer to definitions of ISO 9626 and ISO 7884. The mentioned diameters may relate to an outer diameter of the nozzles.

Preferably, the method comprises a step of setting up an aseptic environment, wherein preparing the substrate to be additive manufacturable, additive manufacturing of the substrate and drying the scaffold body is performed in the aseptic environment. Such aseptic environment can allow for providing the requirement needed for manufacturing drug formulations.

As mentioned above in one preferred embodiment, drying the scaffold body is lyophilizing the scaffold body. Thereby, the method preferably comprises the steps of: gathering lyophilization properties of the specific lyophilization procedure applied when lyophilizing the scaffold body; designing a shape of the scaffold body in accordance with the gathered lyophilization properties; and configuring the additive manufacturing of the substrate such that the scaffold body is formed of the substrate in the designed shape.

Preferably, the substrate is additive manufactured onto a freezing member such that the scaffold body is frozenly formed. In particular, by involving the freezing member, the scaffold body can efficiently be provided as frozen formulation. The freezing member can be a cooled platform or similar structure.

### BRIEF DESCRIPTION OF THE DRAWINGS

The method according to the invention is described in more detail herein below by way of exemplary embodiments and with reference to the attached drawings, in which:

FIG. 1 shows an embodiment of a method according to the invention for generating a first embodiment of a dried drug formulation;

FIG. 2 shows the set up of layers of 3D printing in the method of FIG. 1;

FIG. 3 shows a 3D printer involved in the method of FIG. 1; and

FIG. 4 shows a second embodiment of a dried drug formulation generated by a method according to the invention.

### DESCRIPTION OF EMBODIMENTS

In the following description certain terms are used for reasons of convenience and are not intended to limit the invention. The terms “right”, “left”, “up”, “down”, “under” and “above” refer to directions in the figures. The terminology comprises the explicitly mentioned terms as well as their derivations and terms with a similar meaning. Also, spatially relative terms, such as “beneath”, “below”, “lower”, “above”, “upper”, “proximal”, “distal”, and the like, may be used to describe one element's or feature's relationship to another element or feature as illustrated in the figures. These spatially relative terms are intended to encompass different positions and orientations of the devices in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. The devices may be otherwise oriented (rotated 90 degrees or at other orientations), and the spatially relative descriptors used herein interpreted accordingly. Likewise, descriptions of movement along and around various axes include various special device positions and orientations.

To avoid repetition in the figures and the descriptions of the various aspects and illustrative embodiments, it should be understood that many features are common to many aspects and embodiments. Omission of an aspect from a description or figure does not imply that the aspect is missing from embodiments that incorporate that aspect. Instead, the aspect may have been omitted for clarity and to avoid prolix description. In this context, the following applies to the rest of this description: If, in order to clarify the drawings, a figure contains reference signs which are not explained in the directly associated part of the description, then it is referred to previous or following description sections. Further, for reason of lucidity, if in a drawing not all features of a part are provided with reference signs it is referred to other drawings showing the same part. Like numbers in two or more figures represent the same or similar elements.

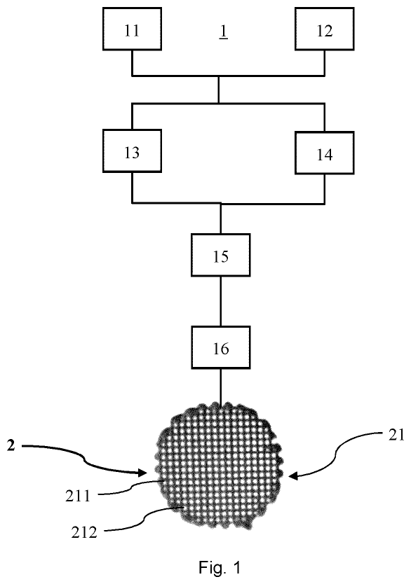

FIG. 1 shows an embodiment of a method 1 according to the invention. In the method 1 a lyophilized drug formulation 2 as dried drug formulation is generated as follows:

In a step 11, characteristics or properties of a fluid substrate containing drug are gathered. For this, the substrate is analysed and information about the drug as well as other components of the substrate are obtained. For example, in analysis of the substrate specific properties such as viscosity, granularity, temperature resistance and the like can be measured.

In parallel, in a step 12, characteristics of the intended additive manufacturing and lyophilization are gathered. For this, the features and capabilities of the devices involved, i.e. a 3D printer 3 and a freeze dryer, are analysed and/or obtained.

In a step 13, an intended shape of the substrate is designed on a computer using a computer aided design software (CAD). In particular, the intended shape comprises a scaffold body. When designing the shape, the gathered characteristics and properties of the substrate, of the 3D printer 3 and of the freeze dryer are considered. Also, further information is considered such as requirements to be met by the final lyophilized substrate. Such requirements may involve, e.g., robustness, visual appearance, residual moisture or the like.

In a step 14, the substrate is prepared. Thereby, the drug and other components such as excipients are composed. The substrate is particularly prepared to be suitable as ink for the 3D printer. Alternatively, in step 14 the prepared substrate is obtained from another process or entity.

In a step 15, a dataset created by or created subsequently to the CAD process in step 13 is transferred to the 3D printer 3 in order that the 3D printer 3 is configured to print the designed shape. Furthermore, process parameters of the 3D printer are adjusted in accordance with the properties of the substrate or are included within the dataset. After configuration, the 3D printer 3 prints the substrate wherein the scaffold body is formed in accordance with the designed shape.

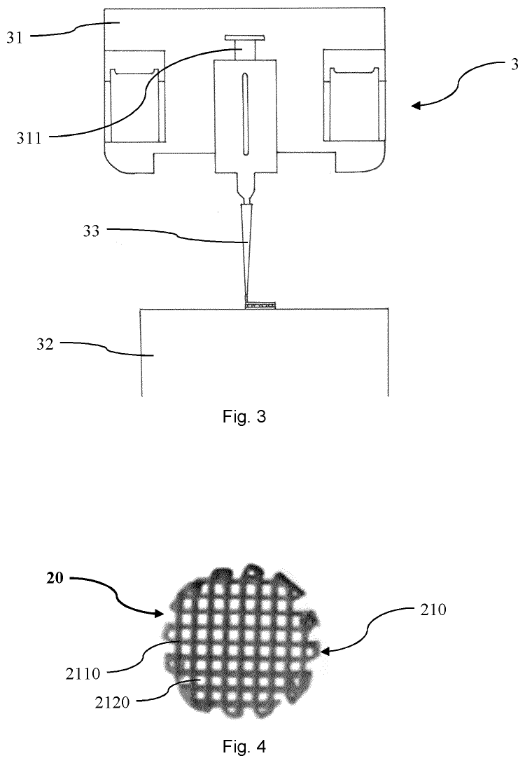

In FIG. 3, the 3D printer 3 involved is schematically depicted. In particular, the 3D printer comprises a printer head 31 and platform 32 on which the substrate is printed. The printer head 31 is equipped with a substrate reservoir 311 in which the fluid substrate is arranged for being processed by the 3D printer 3. The 3D printer 3 further has a nozzle 33 coupled to the substrate reservoir 311. The nozzle 33 is precisely movable such that an accurate printing is possible.

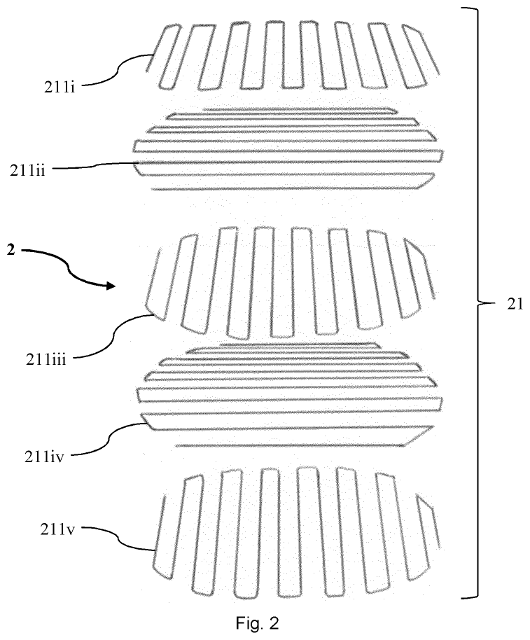

More specifically, as can be seen in FIG. 2, the 3D printer 3 prints the substrate in plural layers 211i-211v shaped to become a frame structure. In each layer, the substrate is printed along a single serpentine line having parallel straight sections and intermediate sections connecting the straight sections. The outer boundary of each serpentine line is essentially circular. Thereby, the first layer 211i, the third layer 211iii and the fifth layer 211v are 90° offset relative to the second layer 211ii and the fourth layer 211iv. Whereas FIG. 4 exemplarily shows five layers, it is understood that there are additional layers provided in the identical way to achieve an appropriate thickness.

Turning back to FIG. 1, in a step 16, the scaffold body made of the substrate is transferred from the 3D printer 3 to the freeze dryer. More specifically, the scaffold body is printed on a large tray that allows the allocation of multiple scaffold bodies or printed individually on smaller trays for single transportation to the freeze dryer or to the desiccation tunnel. Those trays are then positioned inside the freeze dryer. In the freeze dryer, the substrate is dried such that at the end the lyophilized drug formulation 2 results having the previously prepared shape, i.e. the shape of the scaffold body 21.

The method further comprises a step of setting up an aseptic environment. At least the steps 14 to 16 are performed in this aseptic environment.

The scaffold body 21 of the lyophilized drug formulation 2 is cylindrical having a frame structure 211 formed by the printed layers 211i-211v and a plurality of pores 212. The pores 212 are passageways vertically extending through the cylinder as well as passageways horizontally extending though the cylinder. In particular, each passageway is straight and extends through the cylinder opening at two opposing end sides of the cylinder.

In FIG. 4 a second embodiment of a lyophilized drug formulation 20 is shown. It is manufactured in the same or a similar process as the first lyophilized drug formulation 2 described above. The second lyophilized drug formulation 20 has a scaffold body 210 provided with a frame 2110 and straight pores 2120 horizontally and vertically extending through the scaffold body. However, a diameter of the pores 2120 is larger than a diameter of the pores 212 of the first lyophilized drug formulation 2. Like this, another heat transfer, vapor discharge and diluent penetration can be achieved. For example, due to the comparably large pores 2120, the design of the second lyophilized drug formulation 20 allows for a comparably large volume flow to exit the drug formulation 20 which, e.g., allows for dissipating away a comparably large amount of vapor during lyophilisation and also allows a better reconstitution due to larger pores in which the reconstitution media could penetrate the lyophilized drug formulation 20.

This description and the accompanying drawings that illustrate aspects and embodiments of the present invention should not be taken as limiting-the claims defining the protected invention. In other words, while the invention has been illustrated and described in detail in the drawings and foregoing description, such illustration and description are to be considered illustrative or exemplary and not restrictive. Various mechanical, compositional, structural, electrical, and operational changes may be made without departing from the spirit and scope of this description and the claims. In some instances, well-known circuits, structures and techniques have not been shown in detail in order not to obscure the invention. Thus, it will be understood that changes and modifications may be made by those of ordinary skill within the scope and spirit of the following claims. In particular, the present invention covers further embodiments with any combination of features from different embodiments described above and below. For example, it is possible to operate the invention in embodiments as shown in the Figs. wherein desiccation involving a desiccation tunnel is performed instead of lyophilization.

The disclosure also covers all further features shown in the Figs. individually although they may not have been described in the afore or following description. Also, single alternatives of the embodiments described in the figures and the description and single alternatives of features thereof can be disclaimed from the subject matter of the invention or from disclosed subject matter. The disclosure comprises subject matter consisting of the features defined in the claims or the exemplary embodiments as well as subject matter comprising said features.

Furthermore, in the claims the word “comprising” does not exclude other elements or steps, and the indefinite article “a” or “an” does not exclude a plurality. A single unit or step may fulfil the functions of several features recited in the claims. The mere fact that certain measures are recited in mutually different dependent claims does not indicate that a combination of these measures cannot be used to advantage. The terms “essentially”, “about”, “approximately” and the like in connection with an attribute or a value particularly also define exactly the attribute or exactly the value, respectively. The term “about” in the context of a given numerate value or range refers to a value or range that is, e.g., within 20%, within 10%, within 5%, or within 2% of the given value or range. Components described as coupled or connected may be electrically or mechanically directly coupled, or they may be indirectly coupled via one or more intermediate components. Any reference signs in the claims should not be construed as limiting the scope.

1. A method of generating a dried drug formulation, comprising:
preparing a substrate to be additively manufactured, wherein the substrate comprises a drug;
additive manufacturing the substrate such that a scaffold body is formed of the substrate; and
drying the scaffold body.

2. The method of claim 1, wherein the additive manufacturing comprises 3D printing the substrate.

3. The method of claim 2, wherein preparing the substrate comprises generating a printing ink of the substrate suitable for 3D printing.

4. The method of claim 1, wherein the scaffold body has pores.

5. The method of claim 4, wherein the scaffold body has end sides and each of the pores of the scaffold has an opening at one of the end sides.

6. The method of claim 5, wherein the pores of the scaffold body are passageways extending between at least two of the end sides of the scaffold body.

7. The method of claim 1, wherein the scaffold body has a cylindrical shape.

8. The method of claim 1, further comprising:
gathering drying properties of the specific drying procedure applied when drying the scaffold body;
designing a shape of the scaffold body in accordance with the gathered drying properties; and
configuring the additive manufacturing of the substrate such that the scaffold body is formed of the substrate in the designed shape.

9. The method of claim 1, further comprising gathering substrate properties of the substrate.

10. The method of claim 9, further comprising:
designing a shape of the scaffold body in accordance with the gathered substrate properties; and
configuring the additive manufacturing of the substrate such the scaffold body is formed of the substrate in the designed shape.

11. The method of claim 9, further comprising:
selecting a printhead in accordance with the gathered substrate properties; and
configuring the additive manufacturing of the substrate such the scaffold body is formed via the selected printhead.

12. The method of claim 11, wherein the printhead is selected from a group consisting of a pneumatic printhead, a piston printhead, a screw printhead and an electromagnetic droplet printhead.

13. The method of claim 1, further comprising:
setting up an aseptic environment, wherein preparing the substrate to be additively manufactured, additive manufacturing the substrate and drying the scaffold body is performed in the aseptic environment.

14. The method of claim 1, wherein drying the scaffold body is lyophilizing the scaffold body.

15. The method of claim 14, further comprising:
gathering lyophilization properties of the specific lyophilization procedure applied when lyophilizing the scaffold body;
designing a shape of the scaffold body in accordance with the gathered lyophilization properties; and
configuring the additive manufacturing of the substrate such that the scaffold body is formed of the substrate in the designed shape.

16. The method of claim 1, wherein the substrate is additive manufactured onto a freezing member such that the scaffold body is frozenly formed.

17. The method of claim 1, wherein the drug comprises a protein.
